(19) United States
(12) Patent Application Publication
(10) Pub. No.: US 2020/0297991 A1
(43) Pub. Date: Sep. 24, 2020

(54) Device for Treating Endothelial Dysfunction and Related Conditions through Hemodynamics

(71) Applicant: VasoActiv Biomedical Technologies LLC, Tulsa, OK (US)

(72) Inventor: John M. Owen, Tulsa, OK (US)

(21) Appl. No.: 16/827,210

(22) Filed: Mar. 23, 2020

Related U.S. Application Data
(60) Provisional application No. 62/822,539, filed on Mar. 22, 2019.

(51) Int. Cl. A61N 1/04; A61N 1/36

(52) U.S. Cl. CPC A61N 1/0456; A61N 1/0452; A61N 1/36042; A61N 1/0492

(57) ABSTRACT

A device using sequential, overlapping neuromuscular stimulation of the skeletal muscles giving rise to enhanced, pulsatile, wave-form blood flow and greater laminar shear stress.

### CROSS-REFERENCE TO RELATED CASES

This application claims the benefit of U.S. provisional patent application Ser. No. 62/822,539, filed on Mar. 22, 2019, and incorporates such provisional application by reference into this disclosure as if fully set out at this point.

### FIELD OF THE INVENTION

This disclosure relates to systems and methods for medical treatment of a patient with endothelial dysfunction.

### BACKGROUND OF THE INVENTION

The blood circulation system (cardiovascular system) delivers nutrients and oxygen to all cells in the body. It includes the heart and the blood vessels running through the entire body. Arteries carry blood away from the heart; the veins carry it back to the heart. Blood circulation is critical to health maintenance since poor circulation is a contributing factor to many cardiovascular diseases.

Blood acts as a non-Newtonian fluid, which means it is a fluid that does not follow Newton's Law of Viscosity. The viscosity (the measure of a fluid's ability to resist gradual deformation by shear or tensile stresses) of blood is dependent on shear rate, hematocrit, erythrocyte deformability, plasma viscosity, erythrocyte aggregation, and temperature. Shear rates are important factors within the coagulation process of blood. Platelet adhesion and activation are strongly dependent on shear rate.

Normal blood flow occurs in a laminar fashion such that platelets tend to travel in the center of vasculature, thus reducing their contact with the vascular wall. Furthermore, smooth, laminar flow reduces stress on the endothelium. Blood flow constantly mixes the plasma contents, maintaining a fine-tuned balance between pro- and anti-coagulant factors. Dysregulation of blood flow, due to stasis or turbulent flow of blood, can affect all of these processes and thus promote inappropriate coagulation. Stasis of blood can increase contact between platelets and the vascular wall as well as allow localized imbalances in pro- and anti-coagulant factors within the plasma.

Endothelial cells respond to mechanical conditions created by blood flow and the cardiac cycle. As a result of their unique location, endothelial cells experience three primary mechanical forces: pressure, created by the hydrostatic forces of blood within the blood vessel; circumferential stretch or tension, created as a result of defined intercellular connections between the endothelial cells that exert longitudinal forces on the cell during vasomotion; and shear stress, the dragging frictional force created by blood flow. Of these forces, shear stress appears to be a particularly important hemodynamic force because it stimulates the release of vasoactive substances and changes gene expression, cell metabolism, and cell morphology.

What is needed is a system and method for increasing or augmenting the body's natural mechanical stimulation resulting from blood flow and the cardiac cycle, which may be useful for any number of therapies and treatments for a wide range of diseases and health issues.

### SUMMARY OF THE INVENTION

The invention of the present disclosure, in one aspect thereof, comprises a device comprising having a first receptacle operable to receive a first lead to a first pair of treatment electrodes, and a second receptable operable to receive a second lead to a second pair of treatment electrodes. The device also has a microcontroller configured to selectively supply a current to either or both of the first and second receptacles. With the first pair of electrodes applied to a distal location of a patient limb and the second pair of electrodes applied to a proximal location of a patient limb, the microcontroller supplies current to the first receptacle and the second receptacle in a sequential and overlapping manner to produce a sequential tetanic contraction of skeletal muscle in the patient limb from distal to proximal.

In some embodiments, the device includes a third receptacle operable to receive a third lead to a third pair of treatment electrodes, and a fourth receptacle operable to receive a fourth lead to a fourth pair of treatment electrodes. With the third pair of electrodes applied to the limb of the patient more proximally than the second pair of electrodes and the fourth pair of electrodes applied to the limb of the patient more proximally than the third pair, the microcontroller supplies current to the first, second, third and fourth receptacles in a sequential and overlapping manner to produce a sequential tetanic contraction of skeletal muscle in the patient limb from distal to proximal.

The microcontroller may supply current to the receptacles corresponding to adjacent pairs of treatment pads on the patient limb in an overlapping manner. The microcontroller may supply current to the first, second, third, and fourth receptacle simultaneously and/or stop current to the first, second, third, and fourth receptacle simultaneously. In some cases, the microcontroller selectively provides current to the first, second, third and fourth receptacles such that: the most distal pads receive current, then; 250 ms later, the second most distal pads receive current, then; 250 ms later, the third most distal pads receive current while, simultaneously, the current to the first pads is terminated, then; 250 ms later, the fourth most distal pads receive current while simultaneously the current to the second pads is terminated, then; 250 ms later current to the third most distal pads is terminated, then; 250 ms later current to the fourth most distal pads is terminated.

In various embodiments, the first, second, third, and fourth receptacles collectively provide biphasic, symmetrical, rectangular, high-voltage, milliamp waveforms to the limb via the treatment pads. The device may include a control panel configured for adjusting amplitude of current provided via the first receptacle and second receptacle. In some cases, a plurality of amplifiers interpose the microcontroller and the first and second receptacles.

The invention of the present disclosure, in another aspect thereof, comprise a device having a first plurality of electric leads for application via pairs of treatment pads to a first limb of a patient from a distal to a proximal location on the first limb, and a signal generator providing an electrical neuromuscular stimulation to the first plurality of pairs of treatment pads according to a wave-form. The wave-form is applied in a sequential and overlapping manner to the first plurality of pairs of treatment pads such that the electrical neuromuscular stimulation progresses from the distal to the proximal location on the first limb. The wave-form activates a first most distal pair of pads of the first plurality of treatment pads and thereafter activates a second most distal pair of pads of the first plurality of treatment pads while keeping the first most distal pair of pads of the first plurality of treatment pads activated. The wave-form deactivates the first most distal pair of pads of the first plurality of treatment pads when a third most distal pair of pads of the first plurality of treatment pads is activated.

In some embodiments, the device includes a second plurality electric leads for application via pairs of electric treatment pads to a second limb of the patient from a distal to a proximal location on the second limb. The signal generator may provide electrical neuromuscular stimulation to the second plurality of pairs of treatment pads according to the predetermined wave-form. The wave-form may be applied in a sequential and overlapping manner to the second plurality of pairs of treatment pads such that the electrical neuromuscular stimulation progresses from the distal to the proximal location on the second limb. The wave-form may activate a first most distal pair of pads of the second plurality of treatment pads and thereafter activates a second most distal pair of pads of the second plurality of treatment pads while keeping the first most distal pair of pads of the second plurality of treatment pads activated. The wave-form may deactivates the first most distal pair of pads of the second plurality of treatment pads when a third most distal pair of pads of the second plurality of treatment pads is activated.

In some cases, the device includes a third plurality electric leads for application via pairs of electric treatment pads to a third limb of the patient from a distal to a proximal location on the third limb. The signal generator may provide the electrical neuromuscular stimulation to the third plurality of pairs of treatment pads according to the predetermined wave-form. The wave-form may be applied in a sequential and overlapping manner to the third plurality of pairs of treatment pads such that the electrical neuromuscular stimulation progresses from the distal to the proximal location on the third limb. The wave-form may activate a first, most distal pair of pads of the third plurality of treatment pads and thereafter activates a second most distal pair of pads of the third plurality of treatment pads while keeping the first most distal pair of pads of the third plurality of treatment pads activated. The wave-form may deactivate the first most distal pair of pads of the third plurality of treatment pads when a third most distal pair of pads of the third plurality of treatment pads is activated.

In some embodiments, the device includes a fourth plurality of electric leads for application via pairs of electric treatment pads to a fourth limb of the patient from a distal to a proximal location on the fourth limb. The signal generators may provide the electrical neuromuscular stimulation to the fourth plurality of pairs of treatment pads according to the predetermined wave-form. The wave-form may be applied in a sequential and overlapping manner to the fourth plurality of pairs of treatment pads such that the electrical neuromuscular stimulation progresses from the distal to the proximal location on the fourth limb. The wave-form may activate a first most distal pair of pads of the fourth plurality of treatment pads and thereafter activates a second most distal pair of pads of the fourth plurality of treatment pads while keeping the first most distal pair of pads of the fourth plurality of treatment pads activated. The wave-form may deactivate the first most distal pair of pads of the fourth plurality of treatment pads when a third most distal pair of pads of the fourth plurality of treatment pads is activated.

In some cases, with the present embodiment, the first and second limbs are left and right arms, respectively, and the third and fourth limbs are left and right legs, respectively. The wave-form may be applied to the first and third plurality of treatment pads simultaneously, followed by application of the wave-form to the second and fourth treatment pads simultaneously. Application of the wave-form to the first and third plurality of treatment pads may not overlap with application of the wave-form to the second and fourth plurality of treatment pads.

The invention of the present disclosure, in another aspect thereof, comprise a device having a signal generator operable to selectively provide current to a plurality of receptacles capable of inducing tetanic muscle contractions in a patient, and a plurality of leads from the receptacles to pairs of treatment pads that are selectively affixed to the extremities of a patient such that pairs of pads are applied to at least one limb at a plurality of locations on the limb ranging from distal to proximal. The signal generator selectively applies the current inducing tetanic muscle contraction such that the contractions move from distal to proximal on the limb, and selectively applies the current inducing tetanic contractions in a provide biphasic, symmetrical, rectangular waveform.

In some cases the signal generator applies the current to each pair of treatment pads for about 500 ms. The waveform may overlap the pairs of treatment pads such that at least two pairs of treatment pads provide current to the limb except at a start and end of the waveform. In some cases, the signal generator provides a control panel for adjusting an amount of current delivered via the plurality of receptacles.

### BRIEF DESCRIPTION OF THE DRAWINGS

FIG. 1 is a schematic drawing showing a blood vessel and plurality treatment pads prior to activation according to aspects of the present disclosure.

FIG. 2 is a schematic drawing showing the blood vessel and pads of FIG. 1 at initiation of a treatment sequence.

FIG. 3 is a schematic drawing showing the blood vessel and pads of FIG. 1 as treatment continues from FIG. 2.

FIG. 4 is a schematic drawing showing the blood vessel and pads of FIG. 1 as treatment continues from FIG. 3.

FIG. 5 is a schematic drawing showing the blood vessel and pads of FIG. 1 as treatment continues from FIG. 4.

FIG. 6 is a schematic drawing showing the blood vessel and pads of FIG. 1 as treatment continues from FIG. 5.

FIG. 7 is a schematic drawing showing the blood vessel and pads of FIG. 1 as treatment continues from FIG. 6.

FIG. 8 is a drawing of a wave-form stimulation device for providing treatments according to aspects of the present disclosure.

FIG. 9 is a schematic diagram of a wave form stimulation device according to aspects of the present disclosure.

FIG. 10 is a frontal view of a wave form stimulation device according to aspects of the present disclosure.

FIG. 11 is a side view of the device of FIG. 10.

FIG. 12 is a top view of the device of FIG. 10.

FIG. 13 is a stylized representation of a placement of treatment pads for a patient being treated with a wave form stimulation device according to aspects of the present disclosure.

FIG. 14 is a stylized representation of possible placement of treatment pads relative to major circulatory vessels of a patient being treated with a wave form stimulation device according to aspects of the present disclosure.

FIG. 15 is a flow chart illustrating various results of treatment according to systems and methods of the present disclosure.

FIG. 16 is a stylized representation of a cutaway view of a blood vessel illustrating various negative processes in place.

FIG. 17 is another stylized representation of a cutaway view of a blood vessel illustrating various positive changes as a result of application of various embodiments of systems and methods of the present disclosure.

FIG. 18 is a stylized diagram of a human patient leg illustrating possible treatment pad locations and effects beginning with skeletal muscle contraction.

### DETAILED DESCRIPTION OF THE PREFERRED EMBODIMENTS

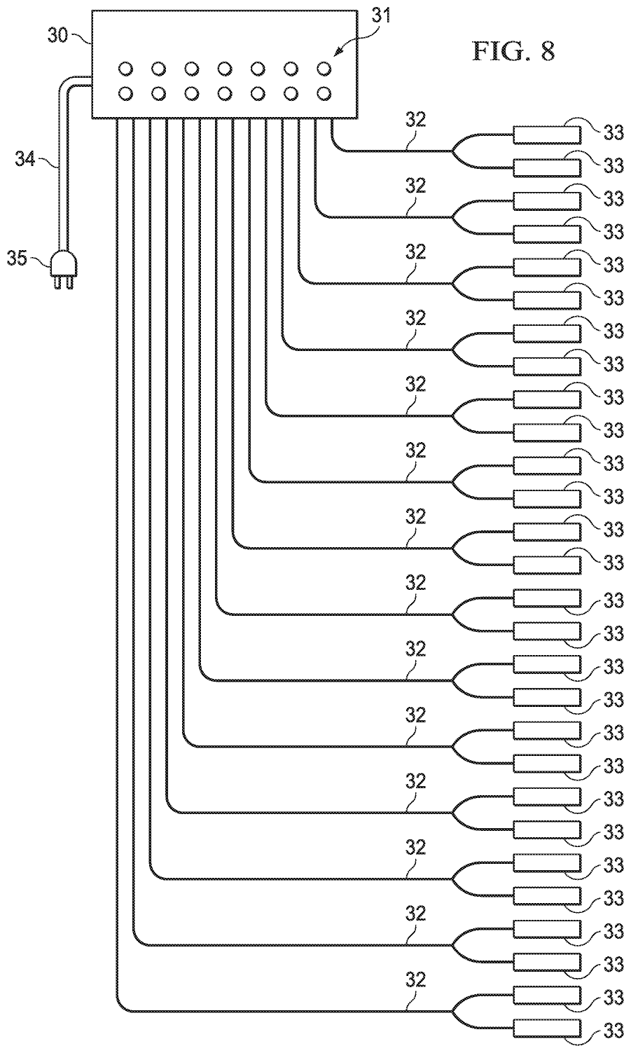

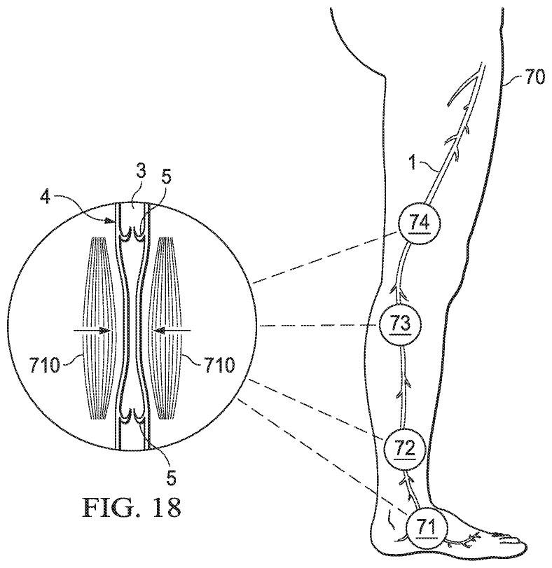

Referring first to FIG. 18, a simplified diagram of a human patient leg or lower extremity 70 is shown. The leg 70 is shown with a large vein 4 running from the foot up to the trunk of the patient. The vein 4 is representative only, but could be the great saphenous vein, for example. As described herein, treatment systems and methods may be based upon placement of various electrode pads (e.g., 33, FIG. 8, discussed below) in pairs to various locations on a patient extremity, trunk, or other location. Electrode pads 33 may be placed on opposite sides of the extremity at the selected location or in another effective location for providing electrostimulation of skeletal muscle according to the methods and techniques described herein.

A location 71 may be a furthest distal location for a pair of pads (e.g., 33) on or near the foot. Location 72 may be superior to the ankle and more proximal than location 71. Location 73 may be just below the knee, for example, and even more proximal than location 72. Location 74 may be superior to the knee and therefore the most proximal location. The locations 71, 72, 73, 74 provide placements for four pairs of pads 33 capable of executing the sequential, overlapping wave form as discussed below. A similar arrangement of pads 33 can be effected for an arm.

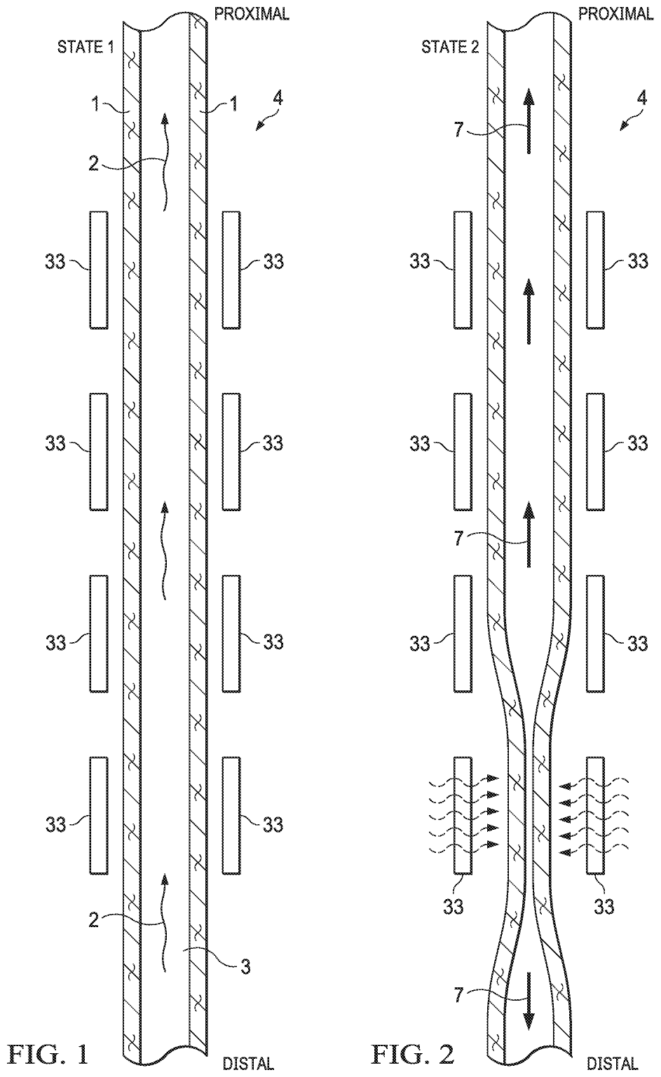

The inset of FIG. 18 shows the anatomical location of skeletal muscle tissue 710 surrounding the blood vessel 4 and contracting under electrical stimulation (e.g., from a pair of opposed treatments pads 33 on the leg). The lumen 3 is thereby squeezed forcing blood away. By sequential activation of the pads as described herein, blood flow can be assured to occur in the proximal direction and back to the torso. It is known that certain large veins within the human body have one-way valves 5 as a part of the anatomy. In some respects, in a healthy individual, such veins can serve to eliminate or reduce “retrograde” flow of blood through the veins which would be in a distal direction (i.e., as blood normally travels in an artery). It should be appreciated that devices and methods of the present disclosure provide therapeutic effect with respect to action upon particular veins whether such veins are those having internal valves or not. Hence, such valves are not illustrated in FIGS. 1-7.

Various embodiments of the present disclosure provide a neuromechanical circulation device (e.g., devices 300, 900, 1000 discussed below) designed to accelerate blood movement as a means of re-establishing physiological levels of blood circulation. Additionally, it is intended to raise laminar shear stress in order to enable endothelial mediated alterations in coagulation, vasodilation, leukocyte and monocyte migration, smooth muscle growth, lipoprotein uptake and metabolism, and endothelial cell survival. The accelerated blood movement triggers a group of events called, collectively, “endothelial mechanotransduction,” thereby upregulating an array of autocrine and paracrine processes that move the vascular system toward homeostasis.

Referring now to FIG. 1, which is a stylized/schematic rendering of a blood vessel 4 with treatment pads 33 in proximity thereto (e.g., placed onto the skin of a patient). For purposes of illustration the blood vessel 4 is shown in the absence of muscle, bone, skin, and the like, though anatomically, skeletal muscle is employed via electrostimulation to, in turn, stimulate or pressurize blood vessels, particularly veins. The blood vessel 4 may be any blood vessel in the body but with respect to particular embodiments of the present disclosure, the blood vessel 4 is a large vein in the foot, leg, hand, or arm, such as a tibial or saphenous vein.

Anatomically, the interior layer of the vein 4 is the endothelial layer 1. This is the innermost layer of a vein that is in actual contact with blood flow 2 and defines the inner flexible lumen 3 of the vein 4. The influence of the endothelium is far reaching and is more than simply a conduit for blood. It is the largest organ in the body and would be equivalent in size to approximately six tennis courts if spread out. It exerts control over an array of mechanisms which serve to maintain vascular tone and blood fluidity by maintaining vascular smooth muscle tone, regulating angiogenesis and cell proliferation, mediating inflammatory and immune responses, regulating vascular permeability, regulating thrombolysis, regulating leukocyte adhesion, regulating platelet adhesion and aggregation, and regulating lipid oxidation, among other actions and effects.

The endothelium exerts such control through endocrine, paracrine and autocrine processes wherein the endothelial cells secrete vasoactive substances such as hormones, genes, proteins, transcription factors and others, resulting in the regulatory actions listed above. This group of events is generally known as, “endothelial mechanotransduction.” Mechanotransduction refers to the processes through which cells sense and respond to mechanical stimuli by converting them to biochemical signals that elicit specific cellular responses.

Endothelial mechanotransduction happens in response to blood flow and laminar shear stress, induced from the mechanical forces caused by the rubbing of blood cells on the endothelium (the lining of blood vessels). When people are young, the normal physiologic levels of blood flow and shear stress keep blood vessels (and the whole cardiovascular system) healthy. Later in life, people make diet and lifestyle choices that can lower blood flow, clog the blood vessels with fatty deposits and impair the regulatory processes necessary for vascular health. The endothelium can then become dysfunctional contributing to atherosclerosis (hardening of the arteries), diabetes, hypertension (high blood pressure), delayed wound healing, vasculitis, congestive heart failure, critical limb ischemia, neuropathy and more.

Systems, devices, and methods of the present disclosure positively affect the endothelium as well as improving vascular return of blood from the extremities of a patient. However, other benefits of aiding return of blood flow not directly related to the endothelium per se may also be observed. Thus, the present disclosure and the effects of the systems, devices and methods herein are not strictly limited to those that rely upon endothelial effects. Further, there may exist in the prior art certain devices and methods that can be observed to improve return blood flow and possibly endothelial function. However, in various embodiments, the present disclosure presents an improved “wave form” that can be applied to a plurality of treatment pads placed on one or more extremities that stimulate blood vessels and the endothelial layer 1 by utilizing the patient's own skeletal muscle as a “pump”. It has been known that such a pumping action is affected by normal movement of a person, particularly in walking, but one who is immobile or otherwise unable to tolerate walking, for example, does not derive the full benefit of this anatomical pump. This pumping action is employed as a treatment for, and preventative measure against, sepsis.

In accordance with embodiments of the present disclosure, electrical stimulation pads 33 may be applied in pairs on opposite sides of a patient's limb. Electrical stimulation applied to the skin can result in contraction of muscle tissue surrounding the vein and provide a pumping action according to the waveforms and methods herein. In reality, many blood vessels may run within any limb or extremity such that one or more veins receive the benefit of the stimulation described herein.

Distal and proximal ends are labelled in FIG. 1. In the case of application of the pads 33 to a patient's leg, for example, the distal end represents the feet and the proximal end represents the upper thigh. As shown in FIG. 1, the endothelial layer 1 and surrounding muscle are relaxed, blood flow 2 is weak through the lumen 3. Four pairs of treatment pads 33 are distributed along the limb from distal to proximal. Treatment pads 33 (also known as electrodes) may be applied to the patient either by self-adhesive means or straps or in a garment. In some embodiments, the pads 33 are applied in pairs, opposed 180° on the feet, calves, lower thighs, and upper thighs. The hands, forearms, biceps, and shoulders, provide other placement locations that may be acceptable and may achieve the desired results. In some embodiments, in order to achieve the desired therapeutic threshold of blood movement, a minimum of 4 pairs of pads on each extremity must be used.

Referring now to FIG. 2, a schematic drawing showing the blood vessel 4 and pads 33 of FIG. 1 at initiation of a treatment sequence. Here, the most distal pair of pads 33 has been activated by application of current resulting in squeezing or closing of a portion of the lumen 3 by surrounding skeletal muscle. Current application is illustrated by arrows flowing through the associated pad 33 (as in the lowermost pair of pads 33 of FIG. 2). As explained, it is the contraction of surrounding skeletal muscle that actual results in contraction of the associated blood vessel 4 (see FIG. 18). For purposes of the present disclosure, it is understood that voltage is also applied, and the particular relationship between applied voltage and applied current may rest upon a number of factors including the impedance of the pads 33 and the patient's body. In some embodiments, voltage may be applied to one pad out of a pair while the opposite pad acts as ground, or is supplied with a negative voltage thereby increasing current flow or voltage differential even further (within safe limits) while limiting the amount of voltage (positive or negative) applied to any single pad. In any event, blood flow 7 may be (or occur, or move) both proximal and distal at this stage, particularly if the vein 4 is a vein without anatomical valves or if the valves are weak or otherwise ineffective.

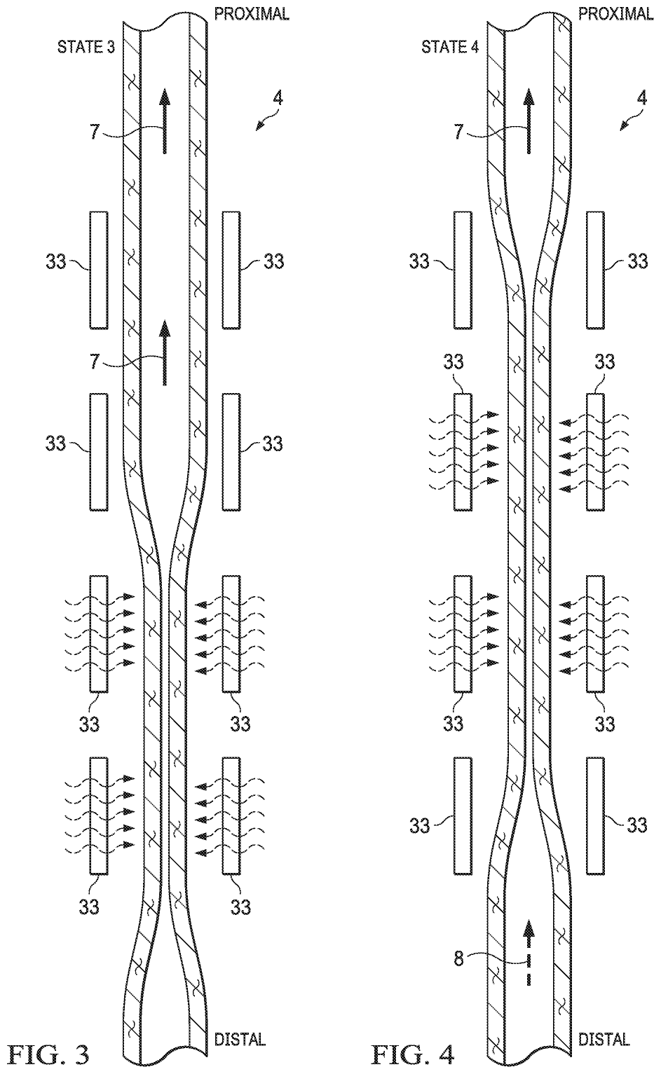

Referring now to FIG. 3 a schematic drawing showing the blood vessel 4 and pads 33 of FIG. 1 as treatment continues from FIG. 2 is shown. Here an overlapping, sequential protocol wave-form, state 3, of the present disclosure can being to be seen. The second most distal pair of pads 5 receive current causing muscle contractions which squeeze the blood vessel 4, closing the lumen 3 and forcing blood flow 7 from the area. None of the blood flow 7 is forced distally since the first pair of pads is still receiving current. Additionally, the blood flow 7 may be more forceful that that experienced at rest. Particularly if the patient is in ill health or non-ambulatory. The blood flow 7 is substantial enough to induce shear stress and activation of the endothelial layer 1 as discussed herein.

Referring now to FIG. 4, a schematic drawing showing the blood vessel and pads of FIG. 1 as treatment continues from FIG. 3 is shown. FIG. 4 shows the showing of the overlapping, sequential protocol, state 4. Here, the third most distal pair of pads 33 receives current causing muscle contractions which squeeze the blood vessel 4, closing the lumen 3 and forcing blood 7 further from the area (in the proximal direction). None, or at least very little, of the blood is forced distally since the second most distal pair of pads 33 (adjacent in the distal direction) is still receiving current. Current to the first pair of pads activated is terminated causing the blood vessel 4 to be allowed to expand and begin drawing refill blood 8 into the lumen 3.

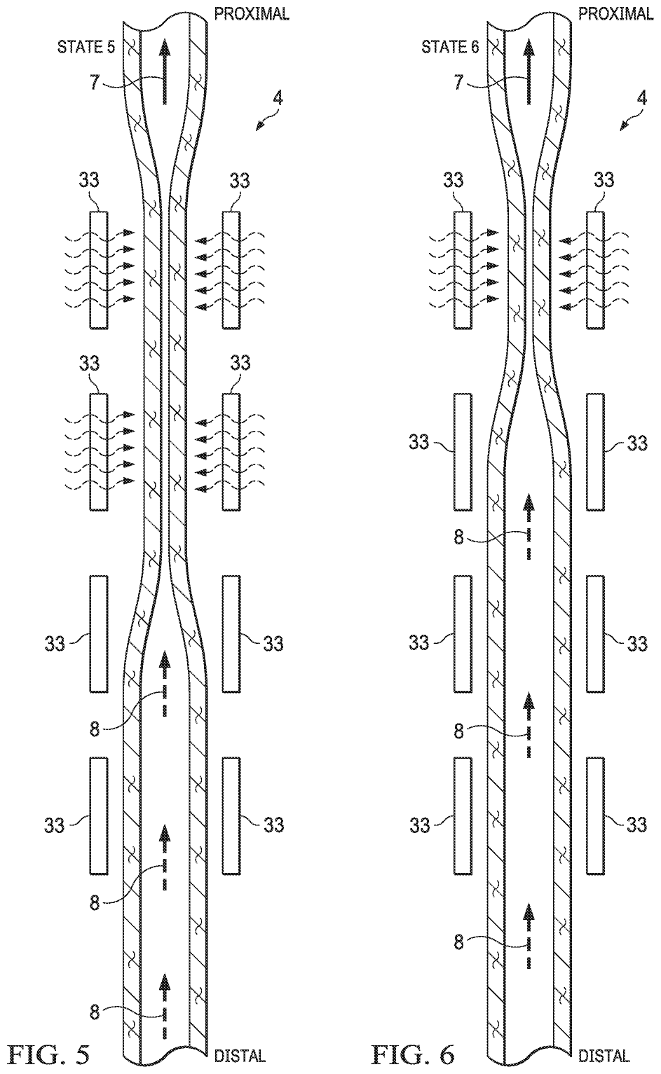

Referring now to FIG. 5, a schematic drawing showing the blood vessel 4 and pads 33 of FIG. 1 as treatment continues from FIG. 4 is shown. FIG. 5 shows the overlapping, sequential protocol, state 5. The fourth most distal pair of pads 33 receive current causing muscle contractions which squeeze the blood vessel 4, closing the lumen 3 and forcing blood 7 from the area further proximally. Again, little or none of the blood 7 is forced distally since the third most distal pair of pads is still receiving current. Current to the second pair of pads (second most distal and also second activated) is terminated after activation of most proximal pair of pads the allowing the blood vessel 4 to expand even further toward the proximal direction and continue to draw refill blood 8 deeper into the lumen 3.

Referring now to FIG. 6, a schematic drawing showing the blood vessel 4 and pads 5 of FIG. 1 as treatment continues from FIG. 5 is shown. FIG. 6 shows the overlapping, sequential protocol, state 6. Current to the third pair of pads 33 (third distally and also third activated) is terminated allowing the blood vessel 4 to expand and continue drawing refill blood 8 deeper into the lumen 3.

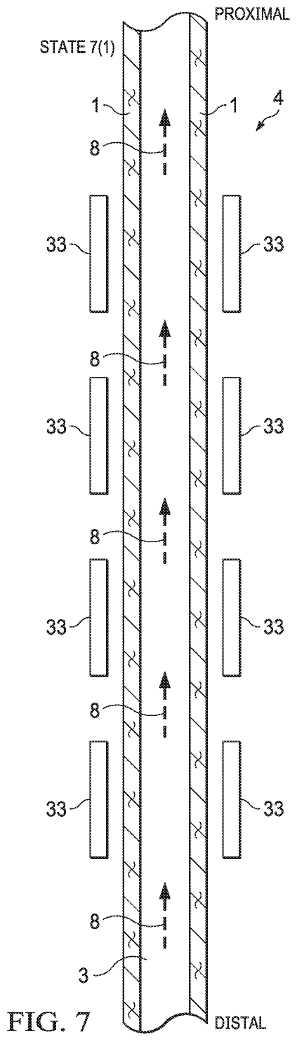

Referring now to FIG. 7, is a schematic drawing showing the blood vessel 4 and pads 33 of FIG. 1 as treatment continues from FIG. 6. Current to the fourth pair of pads 33 (most proximal) is terminated allowing the blood vessel 4 to re-expand along the entire length of the treatment draw refill blood 8 deeper into the lumen 3. This illustrate state, following application of a full wave form through the full set of pads 5 is substantially similar to state 1, FIG. 1. However, blood flow 8 is moving with more force than before (e.g., more forcefully than blood flow 2, FIG. 1). This is the major result of overlapping, sequential timing and the plurality of treatment pads according to embodiments of the present disclosure.

Although the sequence of FIGS. 1-7 illustrates a treatment mode employing four pairs of pads 33, it should be understood that more or fewer pairs of pads 33 may be employed. However, the overlapping aspects of the treatment wave form method would require at least two pairs of pads. Additionally, four pairs as shown provide sufficient stimulation of muscles along a limb so as to enhance proximal blood flow from an extremity to the patient's heart. This is called venous return, and results, according to the Frank-Starling law in higher preload and stroke volume. The wave-form device, system, and method thus raises cardiac output which in many disease states is highly desirable. If, for some reason, further stimulation points are desired, more than four pairs of pads 33 may be provided and it may be possible to activate a second wave-form before the first has completed (if sufficient distance has been provided between them that there is sufficient return blood flow 8 to be “pushed” by a second wave-form).

Referring now to FIG. 8, a drawing of a wave-form stimulation device 30 for providing treatments according to aspects of the present disclosure is shown. The wave-form stimulation device may be, in effect, a signal generator. Thus, it may include all necessary hardware and controls as are known in the art to safely apply various electrical signals, currents and voltages that are therapeutic yet safe for the human body. The wave-form stimulation device 30 comprises a plurality of leads 32. Each lead 32 attaches to a pair of treatment pads 33. An electrical cord 34 and plug 35 for alternating current (AC) input from a wall socket is provided. The treatment device 30 contains the necessarily internal hardware to convert the AC power to direct current (DC) for safe application to the patient via the leads 32 and pads 33. A number of controls 31 including necessary knobs, dials, levers, switches, and the like are provided to enable the operating therapist to control current/voltage applied within safe but therapeutically effective parameters.

In some embodiments, the leads 32 may be divided into groups of four, such that four pairs of pads may be applied to an extremity or limb of a patient. The number of leads 32 may vary. In some embodiments, at least 8 pairs of leads are provided such that both arms or both legs of a patient may have at least four pairs of pads applied in sequence. In another embodiment, 16 pairs of leads are provided such that both arms and both legs may be provided with four pairs of leads and all extremities be subject to the therapeutic application of the electrical wave-forms discussed herein.

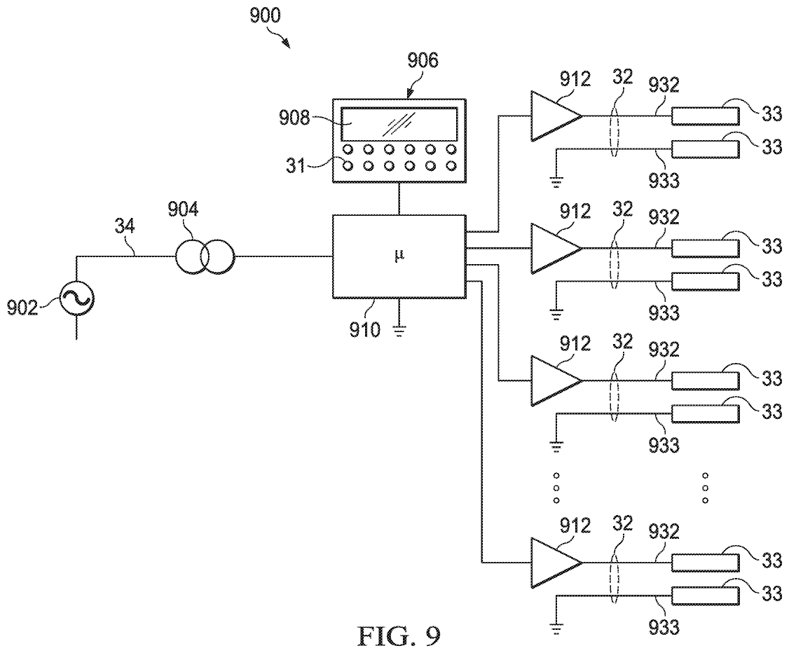

Referring now to FIG. 9 a schematic diagram of a wave form stimulation device 900 according to aspects of the present disclosure is shown. The device 900 represents a simplified internal schematic diagram of the device 30 and other machines suitable for executing the therapies and operations of the present disclosure. It should be understood that additional components such as logic boards, resistors, transistors, capacitors, timing circuits and others known to one of skill in the art may be required or desirable to produce an operational product. However, for simplicity, not every such device as known to one of skill in the art is illustrated. As shown the device 900 may operate from an A/C source 902 such as a 110/220 power outlet operating at 50/60 Hz or another commonly encountered voltage and frequency. Such power source may be transformed to a suitable DC supply by an AC/DC converter 904 as is known in the art.

In some embodiment, a microcontroller 910 provides for execution of a selected program or protocol as well as activation of leads 32 in the required order, and at the required voltage or current to safely induce tetanic skeletal muscle contraction for effecting methods and techniques according to the present disclosure. The microcontroller 910 may be a programmable device such as a commercially available microcontroller or system-on-a-chip device. In other embodiments the microcontroller 910 may be a part of a general-purpose computer or device such as a personal computer, tablet, or other programmable device. The microcontroller 910 may also be provided as a field programmable gate array (FPGA), an application-specific integrated circuit (ASIC), or another suitable device.

The microcontroller 910 may selectively activate various leads 32 according to the methodology described herein. Output from the leads 32 going to the pads 33 may be amplified as represented by amplifiers 912. One pad 33 from each pair associated with a lead 32 may be amplified while the other is grounded, as illustrated. Thus each lead 32 may actually comprise a plurality of one or more leads or traces as shown. Here, an amplified lead 932 is provided along with a ground lead 933 as a subcomponent of each lead 32. In other embodiments, voltages provided between each pair of pads 33 may be based on application of a positive voltage to one pad 33 on a lead 32 and negative voltage on the opposite pad. In such case, additional amplifiers (not shown) may be provided where needed to operate the negative lead at the appropriate voltage. It should be understood that amplified lead 932 may also be operated to provide a negative voltage opposite ground lead 933. For simplicity, the number of leads 32 and pads 33 shown in FIG. 9 is reduced relative to the number that may be provided (see, e.g., FIG. 8).

The microcontroller 910 may be controllable or programmable via console 906 that may have amplitude controllers 31 and other controls provided thereon. Controls that dictate timing, stimulation, treatment length, voltage, amplitude, order of application, and other parameters may be provided. Controls can include buttons, sliders, knobs, switches and any other suitable input mechanism as is known in the art. In some embodiments a screen 908 is provided that displays the mode of operation, treatment time remaining, and other parameters is provided. The screen 908 may comprise a touch screen and may provide a graphical user interface allowing selection of control parameters. All control parameters may be communicated via the controllers 31 and/or screen 908 to the microcontroller 910 for execution. The microcontroller 910 may be provided with programming that prevents entry or execution of ineffective or unsafe parameters. It should be understood that the controls, including controllers 31 and/or screen 908 may have various microcontroller and other control devices associated therewith, although these may still communicate with the microcontroller 910 that controls application of voltage or current to the leads 32.

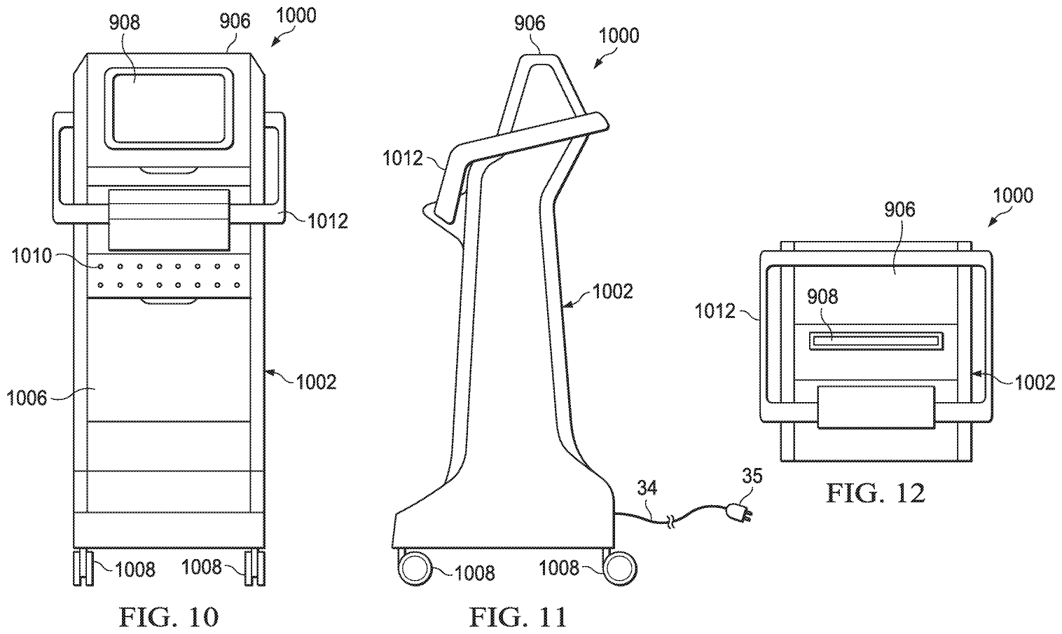

Physically a wave form device 30 (FIG. 8) or device 900 (FIG. 9) as well as other embodiments according to the present disclosure may appear as illustrated in FIGS. 10, 11, and 12. Referring now FIG. 10 a frontal view of a wave form stimulation device 1000 according to aspects of the present disclosure is shown. The illustrated embodiment of the device 1000 is shown in side view in FIG. 10 and in top view in FIG. 11. The device 1000 may comprise a cart 1002 mounted on casters 1008 for ease of movement (e.g., into and out of treatment rooms and the like). The console 906 may be mounted on or near a top of the cart 1002. As illustrated, setup and operation, as well as display or operating parameters, occurs via the touch screen 908 such that no other controls are needed. A graphic user interface may be provided for ease of use.

A panel of receptacles 1010 may be located on the cart 1002 possibly on or near the console 906. The receptacles 1010 selectively receive the leads (e.g., leads 32) for connection from the device 1000 to a patient. The receptacles 1010 may be reusable and may comprise various connectors, sockets, and the like as are known in the art. In this manner, leads 32 may be stored when not in use, or easily replaced when needed. A drawer 34 may be provided for storing the leads 32, pads 33, cleaning supplies, tape or other items when not in use.

In some embodiments, a handle 1012 may be provided for aiding in movement of the device 1000. The electrical cord 34 and plug 35 may extend from the cart 1002 or console at a convenient location. The cord 34 and plug 35 may be retractable or provided with a storage winder, tabs, or retainers for securement for transport or when not in use.

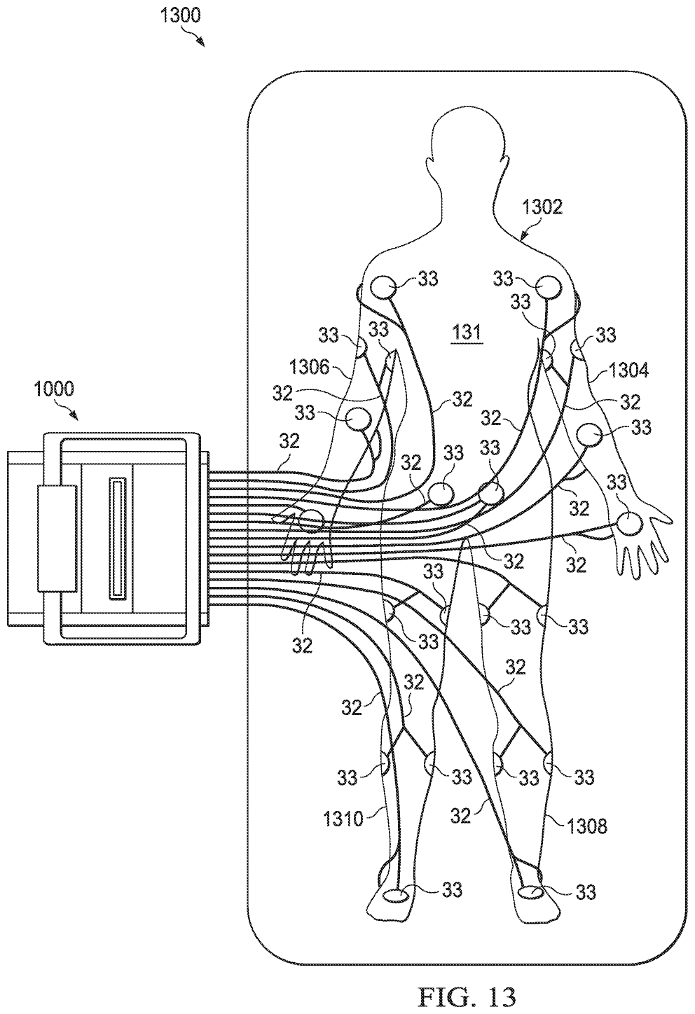

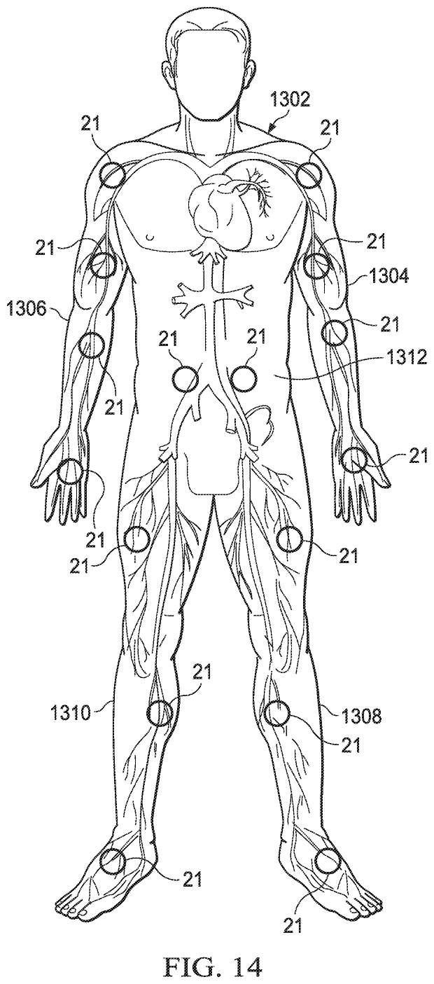

Referring now to FIG. 13 a stylized representation of a placement of treatment pads 33 for a patient 1302 being treated with a wave form stimulation device according to aspects of the present disclosure is shown. The patient 1302 may be placed on a bed 1300 or other suitable location. The waveform stimulation device 100 may be placed in proximity to the patient 1302 with a plurality of leads 32 plugged into receptacles 1010 and then affixed via treatment pads 33 to the patient 1302 in pairs. Four pairs of treatment pads 33 may be applied to the left upper extremity or arm 1304, four pairs of pads 33 may be applied to the right arm 1306, four pairs of pads 33 may be applied to the left lower extremity or leg 1308, and four pairs of pads 33 may be applied to the right leg 1310. The pads 33 may be applied on opposite sides of the limb such that one of each pair of pads 33 is opposed from the other. Other pairs of pads may be applied at other locations on the torso 1312 for simulation of skeletal muscle and blood vessels in the torso 1312.

The device 1000 may apply the sequential, possibly overlapping, wave form to each limb 1304, 1306, 1308, 1310 in the sequence (with respect to each extremity) discussed with respect to FIGS. 1-7. Thus blood may be moved from distally in the extremities to proximal, or back toward the heart and torso. Various orders may be applied to the order in which each limb 1304, 1306, 1308, 1310 receives stimulation. For example, the wave form may be applied one or more times to the left arm 1304, then the right arm 1306, then the left leg 1308, and then the right leg 1310. In some cases, the waveform is applied to more than one limb simultaneously (either wholly simultaneously or with at least some overlap). For example, the left arm 1304 may be treated concurrently with the right leg 1310 followed by the right arm 1306 being treated concurrently with the left leg 1308. It is noted that this activation would correspond to the movement of opposite side arms and legs as in walking. Other limb stimulations and overlap of stimulation is contemplated where therapeutic effects are still seen.

FIG. 14 is a stylized representation of possible placement locations 21 of treatment pads (33, not shown in FIG. 14 for clarity) relative to major circulatory vessels of a patient 1302 being treated with a wave form stimulation device according to aspects of the present disclosure. It should be understood that the locations 21 correspond in some or all cases to where a pair of pads 33 may be applied (180 degree apart, for example). In one embodiment, current is sent to the treatment pads 33 attached to one or more limbs in the following manner:
1. the most distal pads receive current, then
2. ˜250 ms later, the second most distal pads receive current, then
3. ˜250 ms later, the third most distal receive current while, simultaneously, the current to the first pads is terminated.
4. ˜250 ms later, the fourth most distal receive current while simultaneously the current to the second pads is terminated.
5. ˜250 ms later current to the third most distal pads is terminated.
6. ˜250 ms later current to the fourth most distal pads is terminated.

Proper treatment protocols in terms of voltage and current to safely induce a tetanic contraction in skeletal muscle are known in the art. Embodiments of the present disclosure follow such protocols. Either direct current or alternating current may also be used according to embodiments of the present disclosure, with the microcontroller 910 (FIG. 9) and/of the amplifiers 912 be capable of providing either direct or alternating current therapies at frequencies appropriate for use on a patient. Currents may be limited to 20 mA or less, for example. In some cases, current may be limited much lower. Voltages may be higher. In some cases up to 300 V or up to 500 V, but in many cases much less. Enough voltage must be used to overcome the resistance of the skin and other organs while limiting the current (and voltage) to a safe level as is known in the art.

In one treatment protocol the patient 1302 may be placed in a reclining chair, treatment bench, or bed 1300. Treatment pads 33 may be placed as illustrated. The operator or technician may wish to inspect the patient's skin thoroughly to be certain that there are no small abrasions or openings, which could serve as a pathway of current and will become very uncomfortable. If the skin is hairy it may need to be shaved, but this may be done the day before the first stimulation session as shaving can cause points of irritation. An alternative to shaving the day before would be to use scissors to clip the hair as close to the skin as possible, being careful not to abrade the skin. Preparing the skin can be a very important first step in preparation for treatment with shear stress therapy. The skin may be cleaned of any lotion, oils, makeup, and or dead skin before application of the treatment pads 33 but the skin should be dry before the treatment pads are placed.

Once the patient 1302 is prepared and the system (e.g., 30, 900, 1000) attached it may be powered on by the operator, who may slowly increase the amplitude according to protocol or patient comfort. Amplitude is adjustable by the operator starting at a level which elicits no response and gradually increased in amplitude until the patient experiences discomfort, then reduced to a comfort level. Safe levels of voltage/current are known in the art and should stimulation applied should remain within safe bounds (these can be a part of the programming of the device 30, 900, 1000). A stop button or panic button (not shown) may be supplied to the patient which provides a signal to the system (e.g., 30, 900, 1000) to end treatment. Absent this, or following a different protocol, 45 minutes of treatment three times per week may be used.

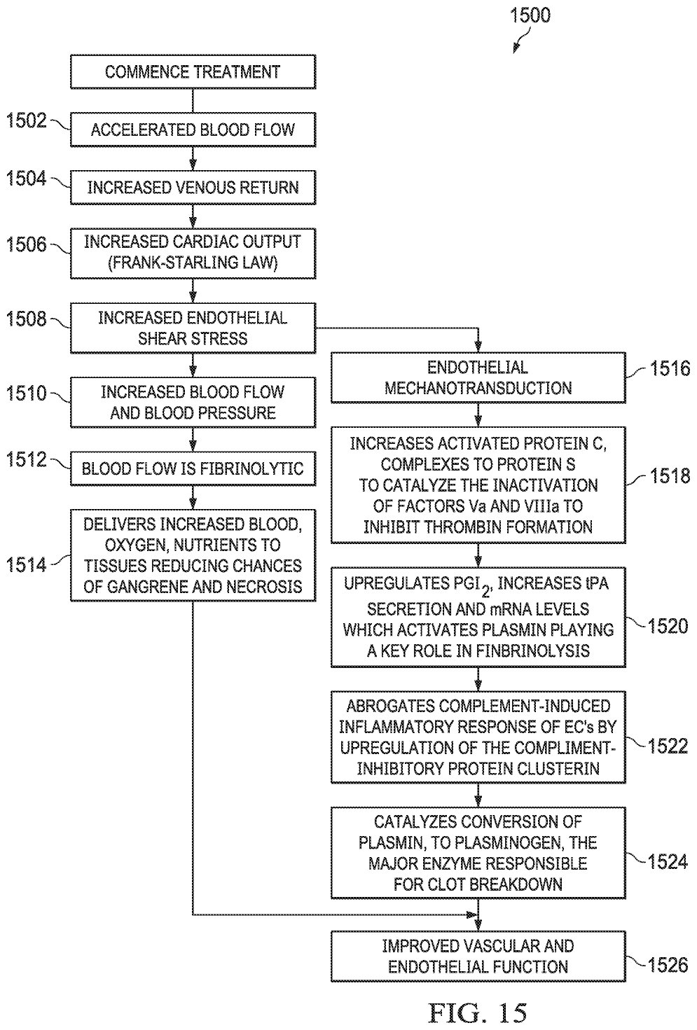

FIG. 15 is a flow chart 1500 illustrating various results of treatment according to systems and methods of the present disclosure. As treatment begins (step 1502) one or all of a number of effects may be observed in the patient. Accelerated blood flow 1502, increased venous return 1504, and increased cardiac output 1506 may be observed as a result of the mechanically induced proximal blood flow resulting from the stimulation from devices of the present disclosure (e.g., 30, 900, 1000).

Increased endothelial shear stress 1508 may be observed as well, which is associated with increased blood flow/pressure 1510, fibrinolytic flow 1512, and deliver of increased blood, oxygen, and nutrients to tissues and organs 1514, reducing chances of gangrene, necrosis, or other disorders, which may also be considered as overall improved vascular and endothelial function 1526.

As a means of invoking endothelial mechanotransduction 1516, devices according to the present disclosure (e.g., 30, 900, 1000) and the systems and methods associated therewith can increase activated protein C as shown at step 1518. This complexes to protein S to catalyze the inactivation of factors Va and VIIIa to inhibit thrombin formation. As shown at step 1520, PGI₂ may be upregulated, which increases tPA secretion and mRNA level thereby possibly activating plasmin which plays a key role in fibrinolysis. Step 1522 shows that the devices and treatment may abrogate complement-induced inflammatory response of endothelial cells by upregulation of the complement-inhibitory protein clusterin. Step 1524 shows that the treatment catalyzes conversion of plasmin to plasminogen, the major enzyme in the body responsible for clot breakdown. Thus, again, as shown at step 1526 overall vascular and endothelial function is improved.

It should be noted that the effects may not be seen in the order shown, nor may all effects be seen in all patients. It should be appreciated that effects may occur simultaneously, or in sequence, though possibly not the sequence shown. Finally, it should be understood that the effect shown are for illustrative purposes only as many other positive effects may also be seen.

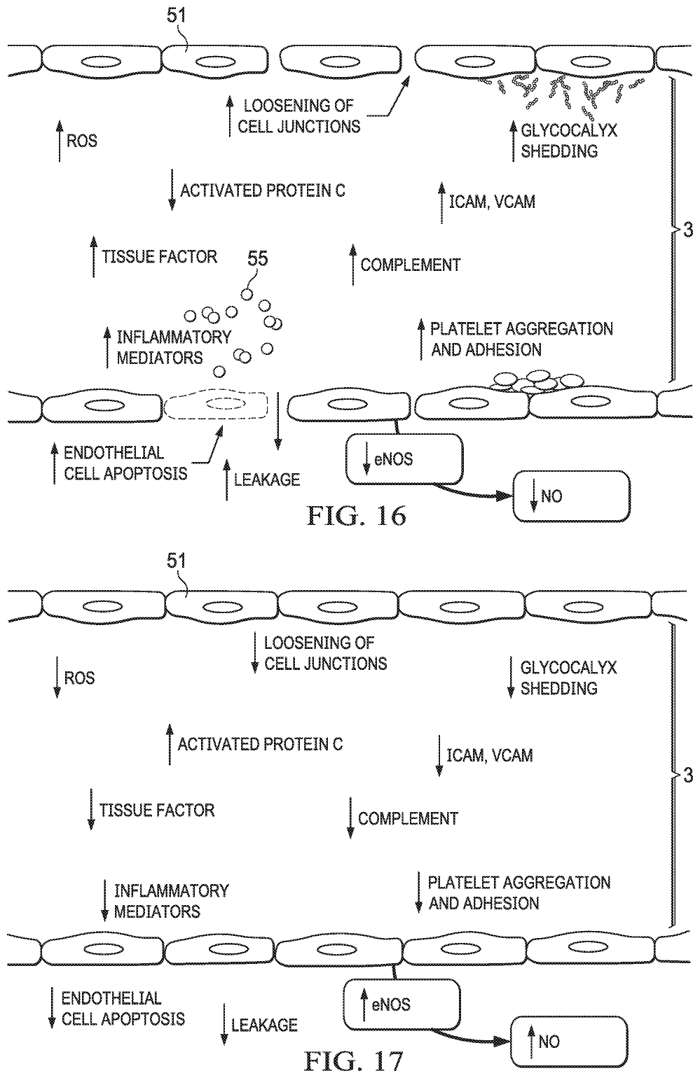

FIG. 16 is a stylized representation of a cutaway view of a blood vessel 4 illustrating various negative processes in place. Upward arrows indicate increase of the associated phenomena owing to disease, lack or movement, or other problems. Down arrows represent reduction in the associated parameter. The processes or conditions shown are exemplary only. Endothelial cells 51 are for illustrative purposes defining the lumen 3 but may not appear as shown. FIG. 17 is another stylized representation of a cutaway view of the blood vessel 4 illustrating various positive changes as a result of application of various embodiments of systems and methods of the present disclosure.

Mechanistically, inflammation modulates thrombotic responses by upregulating procoagulants, downregulating anticoagulants and suppressing fibrinolysis. Shear stress, on the other hand, does the exact opposite: downregulating procoagulants, upregulating anticoagulants and boosting fibrinolysis.

Without treatment reactive oxygen species (ROS), ICAM, VCAM, tissue factors, complements, and inflammatory mediators may also be elevated or increases, but can be seen to decrease with treatment via the devices of the present disclosure. Loosening of cell junctions and leakage from the vessel 4 may be decreased with treatment. Cell death or apoptosis may be decreased with treatment as well as glycocalyx shedding. Platelet aggregation and adhesion within the vessel 4 may be decreased while eNOS and NOS may be increased. Here again, not all positive processes or changes are shown. More or fewer may occur than shown and those that do occur do not necessarily occur simultaneously.

Devices of the present disclosure (e.g., 30, 900, 1000) are designed to accelerate blood movement as a means of re-establishing physiological levels of laminar shear stress in order to enable endothelial-mediated alterations in coagulation, vasodilation, leukocyte and monocyte migration, smooth muscle growth, lipoprotein uptake and metabolism, and endothelial cell survival. The accelerated blood movement triggers a group of events called, collectively, “endothelial mechanotransduction,” thereby upregulating an array of autocrine and paracrine processes that move the vascular system toward homeostasis.

Devices of the present disclosure (e.g., 30, 900, 1000) deliver shear stress as a therapy. Such devices create enhanced, pulsatile, wave-form blood flow in a patient and, through chronic application, to improve vascular health through a return to vascular homeostasis. Biphasic, symmetrical, rectangular, high-voltage, milliamp waveforms are applied to the extremities or elsewhere via the pairs of pads 33 applied to the extremities or elsewhere. The waveforms may have sufficient power to elicit tetanic muscle contractions. The treatment pads 33 may be distributed on all extremities (see., e.g., FIGS. 13-14) with voltage/current delivered in a sequential, overlapping manner to one arm and one leg, from distal to proximal in a cycle lasting about 1.5 seconds then immediately commenced on the contralateral extremities and repeated successively until terminated.

The devices 30, 900, 1000 may automatically stop all treatment after a set period. In one embodiment a treatment is designed to be applied three times per week for 30 to 60 days, at which time the vascular system may be sufficiently restored to maintain vascular homeostasis. Other uses may have different application protocols including frequency and durations of treatment.

In various embodiments, devices 30, 900, 1000 and methods of the present disclosure are is intended to elevate blood flow/circulation and shear stress. The devices 30, 900, 1000 can remodel outward, the vascular architecture through chronic enhanced pulsatile blood movement, and enhanced laminar shear stress and cyclic stretch. The devices 30, 900, 1000 can elevate endothelial mechanotransduction which changes the body chemistry leading to elevated fibrinolytic, angiogenic, antioxidant, and anti-inflammatory substances as well as many other substances. Pressure gradients may be enhanced across capillary beds for better exchange of oxygen and nutrients. Pressure gradients from wound to periwound areas may be enhanced, which promotes angiogenesis and wound healing.

Direct benefits of devices (e.g., 30, 900, 1000), systems, and methods of the present disclosure include movement of blood to block thrombus development, blood movement being thrombolytic in nature. Another direct benefit is restoration the body's natural thrombolytic processes. Increased blood flow helps deliver more oxygen and other nutrients to not only keep the organs viable, but also to increase perfusion and raise tissue oxygen levels throughout the body and thereby prevent, or even reverse, gangrene in the extremities. Restoration of circulation can allow more time for antibiotics and other medicines to work in a sepsis or shock patient with severely compromised circulation. Other direct benefits may also be observed.

Some benefits might be categorized as indirect, although not necessarily less important. Non limiting examples of these indirect benefits include increasing endothelial shear stress which, in turn, triggers a series of physiologic events collectively known as endothelial mechanotransduction, bringing about a cascade of changes to the patient's blood chemistry. The physiologically most important activator of intravascular fibrinolysis is tissue-type plasminogen activator (t-PA). The endothelium synthesizes and stores t-PA and the shear stress dependent release of the enzyme is an important protective response to prevent thrombus formation. Shear stress abrogates the complement-induced proinflammatory response of endothelial cells by upregulation of the complement-inhibitory protein clusterin. Shear stress also decreases endothelial cell tissue factor activity by augmenting secretion of tissue factor pathway inhibitor. It catalyzes the conversion of plasminogen to plasmin, the major enzyme responsible for clot breakdown. It increases activated protein C which complexes to protein S, and they catalyze the inactivation of factors Va and Vlla, thereby serving to inhibit thrombin formation (also known as the protein C anticoagulent cascade). The shear stress also helps restore the body's natural shear- dependent anticoagulant cascades that can reduce endothelial cell dysfunction by rendering the cells less responsive to inflammatory mediators, facilitates the neutralization of inflammatory mediators and decreases loss of endothelial barrier function.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%.

When, in this document, a range is given as “(a first number) to (a second number)” or “(a first number)—(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A device comprising:
a first receptacle operable to receive a first lead to a first pair of treatment electrodes;
a second receptable operable to receive a second lead to a second pair of treatment electrodes;
a microcontroller configured to selectively supply a current to either or both of the first and second receptacles;
wherein, with the first pair of electrodes applied to a distal location of a patient limb and the second pair of electrodes applied to a proximal location of a patient limb, the microcontroller supplies current to the first receptacle and the second receptacle in a sequential and overlapping manner to produce a sequential tetanic contraction of skeletal muscle in the patient limb from distal to proximal.

2. The device of claim 1, further comprising:
a third receptacle operable to receive a third lead to a third pair of treatment electrodes; and
a fourth receptacle operable to receive a fourth lead to a fourth pair of treatment electrodes;
wherein, with the third pair of electrodes applied to the limb of the patient more proximally than the second pair of electrodes and the fourth pair of electrodes applied to the limb of the patient more proximally than the third pair, the microcontroller supplies current to the first, second, third and fourth receptacles in a sequential and overlapping manner to produce a sequential tetanic contraction of skeletal muscle in the patient limb from distal to proximal.

3. The device of claim 2, wherein the microcontroller supplies current to the receptacles corresponding to adjacent pairs of treatment pads on the patient limb in an overlapping manner.

4. The device of claim 2, wherein the microcontroller supplies current to the first, second, third, and fourth receptacle simultaneously.

5. The device of claim 4, wherein the microcontroller stops current to the first, second, third, and fourth receptacle simultaneously.

6. The device of claim 3, wherein the microcontroller selectively provides current to the first, second, third and fourth receptacles such that:
the most distal pads receive current, then;
250 ms later, the second most distal pads receive current, then;
250 ms later, the third most distal pads receive current while, simultaneously, the current to the first pads is terminated, then;
250 ms later, the fourth most distal pads receive current while simultaneously the current to the second pads is terminated, then;
250 ms later current to the third most distal pads is terminated, then;
250 ms later current to the fourth most distal pads is terminated.

7. The device of claim 6, wherein the first, second, third, and fourth receptacles collectively provide biphasic, symmetrical, rectangular, high-voltage, milliamp waveforms to the limb via the treatment pads.

8. The device of claim 1, further comprising a control panel configured for adjusting amplitude of current provided via the first receptacle and second receptacle.

9. The device of claim 1, further comprising a plurality of amplifiers interposing the microcontroller and the first and second receptacles.

10. A device comprising:
a first plurality of electric leads for application via pairs of treatment pads to a first limb of a patient from a distal to a proximal location on the first limb; and
a signal generator providing an electrical neuromuscular stimulation to the first plurality of pairs of treatment pads according to a wave-form;
wherein:
the wave-form is applied in a sequential and overlapping manner to the first plurality of pairs of treatment pads such that the electrical neuromuscular stimulation progresses from the distal to the proximal location on the first limb;
the wave-form activates a first most distal pair of pads of the first plurality of treatment pads and thereafter activates a second most distal pair of pads of the first plurality of treatment pads while keeping the first most distal pair of pads of the first plurality of treatment pads activated; and
the wave-form deactivates the first most distal pair of pads of the first plurality of treatment pads when a third most distal pair of pads of the first plurality of treatment pads is activated.

11. The device of claim 10, further comprising:
a second plurality electric leads for application via pairs of electric treatment pads to a second limb of the patient from a distal to a proximal location on the second limb;
wherein the signal generator provides electrical neuromuscular stimulation to the second plurality of pairs of treatment pads according to the predetermined wave-form;
wherein:
the wave-form is applied in a sequential and overlapping manner to the second plurality of pairs of treatment pads such that the electrical neuromuscular stimulation progresses from the distal to the proximal location on the second limb;
the wave-form activates a first most distal pair of pads of the second plurality of treatment pads and thereafter activates a second most distal pair of pads of the second plurality of treatment pads while keeping the first most distal pair of pads of the second plurality of treatment pads activated; and
the wave-form deactivates the first most distal pair of pads of the second plurality of treatment pads when a third most distal pair of pads of the second plurality of treatment pads is activated.

12. The device of claim 11, further comprising:
a third plurality electric leads for application via pairs of electric treatment pads to a third limb of the patient from a distal to a proximal location on the third limb; and
wherein the signal generator provides the electrical neuromuscular stimulation to the third plurality of pairs of treatment pads according to the predetermined wave-form;
wherein:
the wave-form is applied in a sequential and overlapping manner to the third plurality of pairs of treatment pads such that the electrical neuromuscular stimulation progresses from the distal to the proximal location on the third limb;
the wave-form activates a first, most distal pair of pads of the third plurality of treatment pads and thereafter activates a second most distal pair of pads of the third plurality of treatment pads while keeping the first most distal pair of pads of the third plurality of treatment pads activated; and
the wave-form deactivates the first most distal pair of pads of the third plurality of treatment pads when a third most distal pair of pads of the third plurality of treatment pads is activated.

13. The device of claim 12, further comprising:
a fourth plurality of electric leads for application via pairs of electric treatment pads to a fourth limb of the patient from a distal to a proximal location on the fourth limb; and
wherein the signal generator provides the electrical neuromuscular stimulation to the fourth plurality of pairs of treatment pads according to the predetermined wave-form;
wherein:
the wave-form is applied in a sequential and overlapping manner to the fourth plurality of pairs of treatment pads such that the electrical neuromuscular stimulation progresses from the distal to the proximal location on the fourth limb;
the wave-form activates a first most distal pair of pads of the fourth plurality of treatment pads and thereafter activates a second most distal pair of pads of the fourth plurality of treatment pads while keeping the first most distal pair of pads of the fourth plurality of treatment pads activated; and
the wave-form deactivates the first most distal pair of pads of the fourth plurality of treatment pads when a third most distal pair of pads of the fourth plurality of treatment pads is activated.

14. The device of claim 13, wherein the first and second limbs are left and right arms, respectively, and the third and fourth limbs are left and right legs, respectively.

15. The device of claim 14, wherein the wave-form is applied to the first and third plurality of treatment pads simultaneously, followed by application of the wave-form to the second and fourth treatment pads simultaneously.

16. The device of claim 15, wherein application of the wave-form to the first and third plurality of treatment pads does not overlap with application of the wave-form to the second and fourth plurality of treatment pads.

17. A device comprising:
a signal generator operable to selectively provide current to a plurality of receptacles capable of inducing tetanic muscle contractions in a patient;
a plurality of leads from the receptacles to pairs of treatment pads that are selectively affixed to the extremities of a patient such that pairs of pads are applied to at least one limb at a plurality of locations on the limb ranging from distal to proximal;
wherein the signal generator selectively applies the current inducing tetanic muscle contraction such that the contractions move from distal to proximal on the limb; and
wherein the signal generator selectively applies the current inducing tetanic contractions in a provide biphasic, symmetrical, rectangular waveform.

18. The device of claim 17, wherein the signal generator applies the current to each pair of treatment pads for about 500 ms.

19. The device of claim 17, wherein the waveform overlaps the pairs of treatment pads such that at least two pairs of treatment pads provide current to the limb except at a start and end of the waveform.

20. The device of claim 19, wherein the signal generator provides a control panel for adjusting an amount of current delivered via the plurality of receptacles.
